(19) United States
(12) Patent Application Publication
(10) Pub. No.: US 2024/0087670 A1
(43) Pub. Date: Mar. 14, 2024

(54) SYSTEMS AND METHODS FOR USING MICROBIAL MEASUREMENTS TO DERIVE FINANCIAL PRODUCTS

(71) Applicant: Ancera, Inc., Branford, CT (US)

(72) Inventors: Craig A. Kiebler, Decatur, GA (US); Arjun Ganesan, Newington, CT (US); Charles Judd Copley, Woodbury, CT (US)

(21) Appl. No.: 18/466,639

(22) Filed: Sep. 13, 2023

Related U.S. Application Data
(60) Provisional application No. 63/406,499, filed on Sep. 14, 2022.

(51) Int. Cl. G16B 5/20

(52) U.S. Cl. CPC G16B 5/20

(57) ABSTRACT

Methods and systems for determining a microbial risk assessment for an entity are provided. Microbial data and non-microbial data are received and identified as corresponding to the entity and one or more biological processes associated with the entity. The microbial data and non-microbial data are mapped to one or more risk vectors impacting a state of the one or more biological processes. One or more risk vector ratings are determined for the one or more risk vectors based on the microbial and non-microbial data. A microbial risk assessment including a microbial risk rating for the entity is determined based on the one or more risk vector ratings, where the microbial risk assessment is indicative of a risk of the one or more biological processes to the entity. An expected loss value for the one or more biological processes associated with the entity is determined based on the microbial risk assessment.

### CROSS-REFERENCE TO RELATED APPLICATIONS

This application claims priority to and the benefit of U.S. Provisional Patent Application No. 63/406,499, filed Sep. 14, 2022, and entitled “SYSTEMS AND METHODS FOR USING MICROBIAL MEASUREMENTS TO DERIVE FINANCIAL PRODUCTS”, the entirety of which is incorporated herein by reference.

### TECHNICAL FIELD

The following disclosure is directed to methods and systems for microbial risk assessment, more specifically, methods and systems for assessing and addressing the microbial risk state of entities and affiliates having relationships with the entities based on microbial measurements of the entities' processes.

### BACKGROUND

Microbial characteristics may be used to identify risks corresponding to business operations, physical locations, and other processes (e.g., food production supply chains, crop production, animal (e.g., livestock) health and production, pharmaceutical production, chemical manufacturing, etc.). Such processes may yield one or more outputs (e.g., products) and may be associated with businesses, corporations, organizations, cooperatives, individuals, and other groups (collectively referred to herein as “entities”). Some non-limiting examples of outputs of processes can include food products, animal products, pharmaceutical products, chemical products, manufactured products (e.g., cosmetics, toys, medical devices, etc.), and other products produced by the processes described herein. In some cases, an entity may be an individual or organization associated with (e.g., controlling, managing, or owning) the process. These entities often maintain business relationships (e.g., supply chain relationships) with numerous third-party affiliates (also referred to as “affiliate entities”), such that the microbial characteristics corresponding to affiliates can leave entities vulnerable to risks (e.g., financial and/or business risks) arising from the microbial characteristics of these affiliates' processes. Through use of microbial measurements to identify and quantify risky microbial characteristics, an entity may wish to determine a risk profile (referred to herein as a “microbial risk profile”) corresponding to their operational and biological processes, as well as determine risk profiles corresponding to their affiliates. Further, entities and affiliates may wish to use the determined microbial risk profiles to derive risk mitigation and financial (e.g., risk transfer, valuation, and/or securitization) products, including, for example, insurance, lending, re-insurance, and underwriting products.

### SUMMARY

In some embodiments, microbial measurements may include determining (i) measurements of microbes included in operational and/or biological processes and/or (ii) the biological ‘state’ of processes relating to an animal, herd, crop, location, product, manufacturing (e.g., food production), or supply chain (referred to herein collectively as “process” or “processes”). Such microbial measurements may be indicative of a state and/or an outcome of a particular process, where microbial measurements can be used to predict a process's outcome (e.g., yield for an output) and to identify a process's risks (e.g., disease, loss). For example, particular (e.g., load) levels of parasite eggs in animal feces can cause operational inefficiencies and financial losses related to livestock production, as well as precede severe disease in certain animals, which can result in an increased mortality rate for a livestock producer. Accordingly, correlation, causation, and/or any other suitable relationship between microbial measurements and adverse events in business, operational and/or biological processes may be used to evaluate and quantify operational efficiency, business risks, and identifying business interruptions for entities and their affiliates.

Thus, there exists a current need for a microbial risk assessment technique and supporting systems for (i) determining microbial risk assessments for entities and their affiliates based on microbial measurements of their associated processes (including, for example, biological and/or operational processes, each referred to herein generally as a “process”); and (ii) deriving financial models corresponding to the microbial risk assessments for the entities and their affiliates. In some embodiments, one or more microbial measurements (e.g., data) corresponding one or more processes may be analyzed in isolation or combination with non-microbial data (e.g., environmental conditions) to determine a microbial risk assessment (e.g., a visual score card, numeric value, a written report, and/or other rating method) for an entity. In some cases, the microbial risk assessment may include a rating (e.g., a grade, score, and/or any suitable state indicator) that is indicative of a state of the entity's microbial risk in their processes. The rating may be qualitative or quantitative. For example, the microbial risk assessment may include a standardized rating (e.g., standardized microbial risk rating) that may indicate the entity's microbial risk relative to other entities and/or the entity's affiliates. Such a microbial risk assessment may be used to calculate a predicted loss, and, as a result, derive one or more financial products, including, for example, risk transfer products associated with an entity's processes. In some cases, financial product types and pricing may be offered to entities and their affiliates based on their associated microbial risk assessments and/or predicted loss calculations. For example, an affiliate of an entity identified as having a high level of microbial risk for their processes may not be offered risk transfer (e.g., insurance) products by the entity, thereby enabling the entity to reduce the costs of risk transfer products offered to their other, less-risky affiliates.

In one aspect, the disclosure features a computer-implemented method for assessing and addressing the microbial risk state of entities and affiliates, including determining a microbial risk assessment for an entity. The method can include receiving, from one or more computing systems, microbial data and non-microbial data. The method can include identifying the microbial data and the non-microbial data as corresponding to the entity and one or more biological processes associated with the entity. The method can include mapping the microbial data and the non-microbial data to one or more risk vectors corresponding to the one or more biological processes, wherein each of the one or more risk vectors impact a state of a biological process of the one or more biological processes. The method can include determining, based on the microbial and non-microbial data, one or more risk vector ratings for the one or more risk vectors, each of the one or more risk vector ratings being mapped to a respective risk vector of the one or more risk vectors. The method can include determining, based on the one or more risk vector ratings, the microbial risk assessment for the entity, wherein the microbial risk assessment comprises a microbial risk rating determined based on the one or more risk vector ratings, wherein the microbial risk assessment is indicative of a risk of the one or more biological processes to the entity. The method can include determining, based on the microbial risk assessment, an expected loss value for the one or more biological processes associated with the entity.

Various embodiments of the method can include one or more of the following features. The microbial data can include data collected from at least one of: direct or indirect quantification, culture, sequencing, most-probable-number, secretion assays, polymerase chain reaction (PCR), PIPER, immunoassays, whole genome sequencing, metagenomic sequencing, and next generation sequencing. The microbial data can be sampled at a first time and a second time based on a microbial lifecycle corresponding to the one or more biological processes. In some cases, the microbial data can include microbial load data for Coccidia. In some cases, the microbial data can include microbial serotype data for Salmonella. The non-microbial data can include at least one of: physical structure data for the one or more biological processes, temporal data, spatial data, business operations and financial data, imagery and remote sensing data, economic data, management practices for the one or more biological processes, sanitation practices for the one or more biological processes, pathogen control program data for the one or more biological processes, data corresponding to an output of the one or more biological processes, efficiency data for the one or more biological processes, and environmental data for the one or more biological processes.

In some embodiments, the one or more biological processes can include at least one of: a food production supply chain process, a crop production process, an animal production process, a pharmaceutical production process, a chemical manufacturing process, a feed production process, and a medical device manufacturing process. In some cases, the animal production process can include a poultry production process. In some cases, the one or more risk vectors can include at least one of: a microbial load, a rate of change of the microbial load, a microbial species, a microbial subtype, a microbial serotype, a microbial strain, comorbidities of an output corresponding to the one or more biological processes, efficacy of pathogen control programs for the one or more biological processes, environmental data for the one or more biological processes, efficiency data for the one or more biological processes, historical performance data for the one or more biological processes, economic data, management practices for the one or more biological processes, and an affiliate microbial risk rating for an affiliate entity having a business relationship with the entity. Each of the risk vector ratings can indicate a risk of the respective risk vector to at least one of the one or more biological processes.

In some embodiments, determining the one or more risk vector ratings for the one or more risk vectors can further include at least one of: (i) applying a transformation to the microbial data and/or the non-microbial data to determine a first risk vector rating of the one or more risk vector ratings; and (ii) providing the microbial data and/or the non-microbial data as an input to a model configured to determine a second risk vector rating of the one or more risk vector rating. In some cases, the transformation can include a normalization technique. In some cases, the model can include a partially pooled Bayesian model, and the method can further include determining the second risk vector rating of the one or more risk vector ratings by providing the microbial data as an input to the partially pooled Bayesian model, wherein the microbial data corresponds to an expected statistical distribution. In some cases, the model can include a categorical assessment model, and the method can further include: mapping the microbial data to one or more of a plurality of categorical risk tiers of the categorical assessment model, each of the plurality of categorical risk tiers corresponding to a respective weight factor; and determining the second risk vector rating of the one or more risk vector ratings based on the microbial data and the weight factors. The method can further include modifying the weight factors based on non-microbial data.

In some embodiments, at least one of the microbial data and non-microbial data can include time-series data, and the determining the one or more risk vector ratings for the one or more risk vectors can further include generating, based on the time-series data, a time-series of risk vector ratings for the one or more risk vectors. In some cases, the determining the microbial risk assessment for the entity can further include generating, based on the time-series of risk vector ratings, a time-series of microbial risk ratings, wherein the time-series of microbial risk ratings includes the microbial risk rating. In some cases, the determining the expected loss value for the entity can further include providing the microbial risk rating as an input to loss calculation model configured to generate the expected loss value, where the loss calculation model comprises at least one of: an unaided learning method, a Bayesian predictive function, a non-Bayesian predictive function, and an artificial intelligence technique.

In some embodiments, a financial product is derived from the expected loss value, wherein the financial product can include at least one of: a risk transfer product, a credit product, a derivative product, a valuation product, and a securitization product. The method can further include generating a written report comprising the microbial risk assessment. The method can further include providing the microbial risk assessment via a user interface and/or causing sending of a message comprising the microbial risk assessment. The method can further include causing sending of at least one of the microbial risk assessment or the expected loss value to an affiliate entity having a business relationship with the entity.

In another aspect, the invention features a system for assessing and addressing the microbial risk state of entities and affiliates, including determining a microbial risk assessment for an entity. The system can include corresponding computer systems, apparatus, and computer programs recorded on one or more computer storage devices, each configured to perform the actions of the method. A system of one or more computers can be configured to perform particular actions by virtue of having software, firmware, hardware, or a combination of them installed on the system (e.g., instructions stored in one or more storage devices) that in operation causes or cause the system to perform the actions. One or more computer programs can be configured to perform particular actions by virtue of including instructions that, when executed by data processing apparatus, cause the apparatus to perform the actions.

The above and other preferred features, including various novel details of implementation and combination of events, will now be more particularly described with reference to the accompanying figures and pointed out in the claims. It will be understood that the particular methods and systems described herein are shown by way of illustration only and not as limitations. As will be understood by those skilled in the art, the principles and features described herein may be employed in various and numerous embodiments without departing from the scope of any of the present inventions. As can be appreciated from foregoing and following description, each and every feature described herein, and each and every combination of two or more such features, is included within the scope of the present disclosure provided that the features included in such a combination are not mutually inconsistent. In addition, any feature or combination of features may be specifically excluded from any embodiment of any of the present inventions.

The foregoing Summary, including the description of some embodiments, motivations therefore, and/or advantages thereof, is intended to assist the reader in understanding the present disclosure, and does not in any way limit the scope of any of the claims.

### BRIEF DESCRIPTION OF THE DRAWINGS

FIG. 1 is a block diagram of a microbial risk assessment system, according to some embodiments.

FIG. 2 shows a flowchart of a method for microbial risk assessment, according to some embodiments.

FIG. 3 is a block diagram of an example computer system that may be used in implementing the technology described herein.

### DETAILED DESCRIPTION

The present disclosure is directed to methods and systems for microbial risk assessment and loss prediction and calculation, more specifically, methods and systems for assessing the microbial risk state of entities and affiliates having relationships with the entities based on microbial measurements of the entities' assets, processes, and systems. A microbial risk assessment (e.g., including an associated rating) and loss calculation may be used to derive financial products for the entity corresponding to the microbial risk assessment.

In some embodiments, a microbial risk assessment system may aggregate microbial data and non-microbial data (e.g., temporal data, spatial data, environmental data, etc.) corresponding to one or more biological, business, and/or operational processes each corresponding to one or more entities. In some cases, microbial data and/or non-microbial data can be aggregated at one or more hierarchical levels of a process. For example, microbial data and/or non-microbial data corresponding to a lower level, particular house of a number of houses of a poultry production facility may be used individually or with other microbial and/or non-microbial data to determine a microbial risk profile for an entity. Some non-limiting examples of microbial data may include data collected from and/or using direct or indirect quantification, culture, sequencing, most-probable-number, secretion assays, as well as data collected using polymerase chain reaction (PCR) and its variants, PIPER, immunoassays, whole genome sequencing, metagenomic sequencing, next generation sequencing, and public and private data sources historical lab reports. In some cases, microbial data for a particular entity can include microbial data corresponding to process(es) of one or more of the entity's affiliates and microbial risk ratings of one or more of an entity's affiliates. Some non-limiting examples of non-microbial data may include physical structure data and accompanying descriptors (e.g., water sources, ventilation, lighting, humidity, temperature, bedding, age, condition, living dimensions, feeders, pens, etc.), temporal (e.g., seasonal, time of year, etc.) data, spatial (e.g., geographic, location, elevation, vegetation, latitude and longitude, etc.) data, business operations and financial data, imagery and remote sensing data, market pricing, process management practices, sanitation practices, pathogen control programs and treatments for outputs and/or environments corresponding to outputs, output-specific characteristic data for a process (e.g., final target output size, output growth rates, output mortality, breed/species of the output, etc.), production efficiency data (e.g., feed conversion rates, feed consumption, feed costs, average daily weight gain, total pounds of output produced, grower rank, mortality rate, cost per unit of live weight produced, etc.), and environmental (e.g., physical hazard, soil, weather, emissions and pollution) data. In some cases, management practices for a particular process may be managed by entity and/or individual corresponding to the entity (e.g., a manager, farmer, etc.) In some cases, non-microbial data for a particular entity can include non-microbial data corresponding to process(es) of one or more of the entity's affiliates. In some cases, microbial and/or non-microbial data may be sampled from an output (e.g., product) and/or an intermediate product yielded by a process based on one or more sampling techniques as described herein. Based on the aggregated microbial data and non-microbial data, the microbial risk assessment and loss estimate system may characterize the aggregated microbial data and non-microbial data as corresponding to one or more processes, where each process corresponds to at least one particular entity. Some non-limiting examples of types of processes may include crop (e.g., row crops, leafy greens, vegetables, feed, oils) health and production, animal (e.g., livestock, meat, poultry, aquaculture) health and production, mixed crop-livestock production, consumer packaged goods production (e.g., cosmetics, toys, etc.), sanitation processes, manufacturing processes (e.g., medical device manufacturing, feed production processes), chemical production processes, pharmaceutical production processes, etc.

Based on characterizing the aggregated microbial data and non-microbial data and using the characterized data, the microbial risk assessment system may determine a microbial risk assessment for each entity associated with a process and projected loss estimates based on the microbial risk assessment. In some cases, the microbial risk assessment system may determine a microbial risk assessment for a particular entity based on combination of one or more processes corresponding to the entity. One or more of the microbial risk assessment(s) corresponding to an entity (or an entity's affiliates) may be used to derive one or more financial products (e.g., using loss calculation estimates). Some non-limiting examples of financial products may include risk transfer products (e.g., such as those used in insurance, lending, re-insurance, and underwriting industries) business and/or product valuations, and/or securitizations. Some additional examples of uses for the one or more microbial risk assessments and loss estimates may include factoring, growers access, systems automation, volatility control, derivatives (e.g., call option, European style options, American style options, binary options, etc.), buy now pay later, installments, etc.

As used herein, an “affiliate” of a particular entity may be any individual, organization, corporation and/or other entity that interacts with, provides services to, and/or otherwise has a relationship (e.g., business relationship) with the particular entity.

As used herein, the “criticality” of an entity's relationship to an affiliate may be a measurement or characterization of the extent to which the entity's well-being (e.g., operational integrity, health, reputation, financial position, etc.) is sensitive to (e.g., dependent on) the well-being of the affiliate's process(es), the frequency of such interactions, and/or the volume and/or value of output(s) (e.g., products) associated with the affiliate's process(es).

An entity may monitor the microbial risk status (e.g., risk to processes based on microbial and/or non-microbial data) of one or more of the entity's affiliates. The monitored affiliates may be referred to herein as the entity's “portfolio” of affiliates. An entity's portfolio may include any number of the entity's affiliates (e.g., one or more, dozens, hundreds, thousands, etc.).

“Characteristics” of an entity (e.g., an affiliate or other entity) may include, without limitation, size (e.g., the number of employees or other members of the entity, the entity's market capitalization or annual revenues, number and size of facilities, etc.); the business sector (e.g., industry, sub-industry, etc.) in which the entity operates (e.g., legal services, technology, finance, etc.); age; rate of growth; North American Industry Classification System (NAICS) code; Standard Industrial Classification (SIC) code; a number of services provided by the entity; a microbial risk rating (e.g., as included in a microbial risk assessment as described herein); a geographical location of the entity; one or more microbial risk types of an entity; a number and type of processes of an entity; a geographic location of the processes of an entity; and/or known competitors or entities similar to the particular entity. Values for one or more of the above-listed entity characteristics may be provided by the entity itself, obtained from third-party sources, and/or collected or extracted from publicly available information. In some embodiments, the values for one or more entity characteristics can be stored in a database.

A “microbial risk profile” of an entity may reflect the past, present, and/or future microbial risk characteristics of an entity based on the processes associated with the entity. In some cases, a microbial risk profile of an entity may be based on a microbial risk profiles corresponding to one or more of the entity's affiliates. Such risks may be operational (e.g., forced closure, process improvement, etc.), financial (e.g., loss of revenue, lower profit margins, etc.) and/or perceptive (e.g., brand reputation, etc.). Such a microbial risk profile may be used to calculate a predicted loss calculation. In some embodiments, the microbial risk profile may reflect microbial and non-microbial (e.g., environmental, market, brand, etc.) risks to which the entity and the entity's processes are exposed balanced by the countermeasures that the entity has taken or can take to mitigate the microbial risk. As referred to herein, a microbial risk profile of an entity can include a “microbial risk rating” (also referred to as a “microbial risk score”) for the entity. A microbial risk rating may be quantitative or qualitative. For example, a quantitative microbial risk rating may be expressed as a number within a predetermined range. As referred to herein, an assessment (e.g., individual, periodic, or continuous assessment) of a microbial risk profile of an entity may be known as a microbial risk assessment.

As used herein, “monitoring” an affiliate may refer to determining (e.g., obtaining) a microbial risk assessment of the affiliate from time to time, identifying one or more activities or events relevant to the affiliate's microbial risk and estimated potential financial loss calculations resulting from such risks.

### Some Embodiments of a Microbial Risk Assessment System

As described herein, microbial measurements (e.g., data) corresponding to a particular process can be used to assess (e.g., predict) the process's outcome and to identify the process's risks. Accordingly, correlation and/or causation between microbial measurements and adverse events in processes may be used as proxies and/or direct measures for evaluating operational efficiency, business risks, and identifying business interruptions for entities and their affiliates that are associated with the particular process. For example, microbial measurements indicative of high levels of spoilage organisms in a particular product produced by an entity's process may be indicative of a low yield or reduced shelf-life (e.g., due to spoilage) for the product. A microbial risk assessment corresponding to the entity may account for the expected low yield and/or reduced shelf life for the product, such that a microbial risk rating is low and/or poor relative to other entities associated with similar processes and an associated loss estimate can be calculated. Accordingly, affiliates (e.g., organizations in a supply chain corresponding to the product, financial organizations offering risk transfer products, etc.) of the entity may desire am assessment of the entity's microbial risk assessment (and microbial risk rating) to assess the business risk for maintaining a business relationship with the entity.

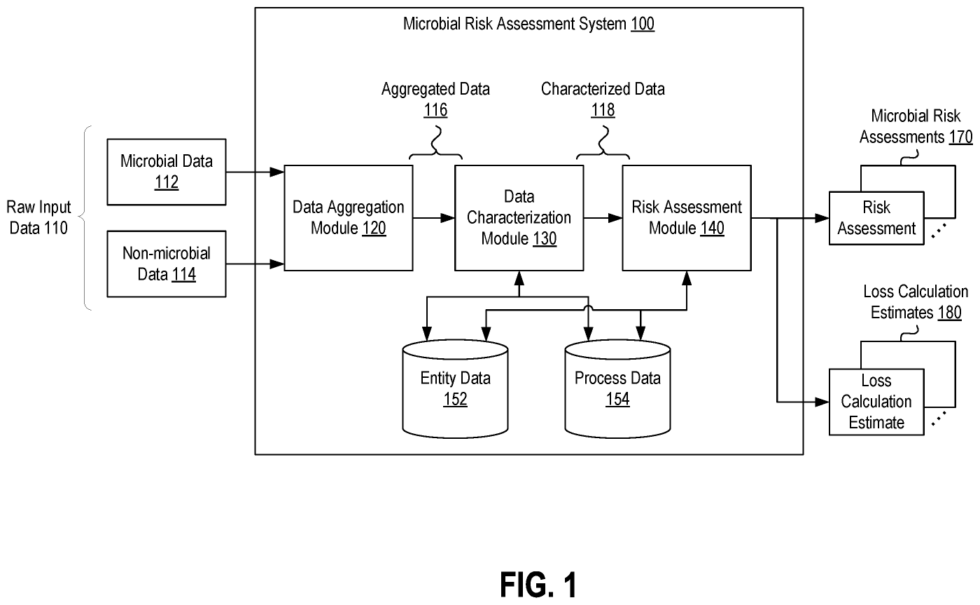

Thus, a system and method for assessing the microbial risk (and related non-microbial risk) for processes associated with entities and their various affiliates is needed. Referring to FIG. 1, an example of a microbial risk management system 100 is shown, according to some embodiments. In some embodiments, the microbial risk management system 100 may determine an assessment (e.g., microbial risk assessment) of a microbial risk profile for one or more entities. A microbial risk assessment may be indicative of a particular entity's microbial and non-microbial risk for one or more processes managed and/or otherwise associated with the entity. The microbial risk management system 100 may receive one or more raw input data 110 as an input and may output one or more microbial risk assessments 170, wherein each microbial risk assessment 170 corresponds to one or more processes associated with a particular entity and may be provided to the entity and/or the entity's affiliates. As an example, the entity and/or entity's affiliates may use the microbial risk assessment 170 for any one of executive board reporting, vendor risk assessment, vendor risk management, self-assessments, compliance mapping, improving risk models in forms of insurance, mergers and acquisitions, and cost of capital calculations. Further, an entity may use the microbial risk assessment 170 to identify areas (e.g., metrics) in a process that may need improvement.

In some embodiments, the microbial risk assessment system 100 may include a data aggregation module 120, a data characterization module 130, and/or a risk assessment module 140. In some cases, the microbial risk assessment system 100 may be communicatively coupled to a third-party computing system. In some cases, the microbial risk assessment system 100 may be coupled to more than one third-party computing system. The microbial risk assessment system 100 may receive one or more raw input data 110. The raw input data 110 may include one or more microbial data 112 and/or one or more non-microbial data 114. Using the raw input data 110, the microbial risk assessment system 100 may characterize the raw input data 110 and determine one or more microbial risk assessments 170 and/or one or more loss calculation estimates 180 (also referred to as “expected loss values”).

In some embodiments, the microbial risk assessment system 100 may be communicatively coupled to one or more internal and/or external (e.g., third-party) data stores. The microbial risk assessment system 100 may include and/or be coupled to (e.g., communicatively connected to) an entity data store 152 and/or a process data store 154. The entity data store 152 may include characteristics associated with one or more entities and/or one or more of each entity's affiliates as described above. The process data store 154 may include one or more types of processes that may be associated with a particular entity as described herein. The process data store 154 may include contextual information for one or more types of processes, such as, for example, one or more indications of microbial data 112 and/or non-microbial data 114 that may be used to provide an indication of a state (e.g., measure the success and/or failure) of each of the types of the processes. In some cases, each indication of a microbial data 112 and/or non-microbial data 114 may correspond to one or more thresholds and/or ranges (e.g., used to determine risk vector ratings) that may be indicative of the state (e.g., success or failure) of a particular process. For example, the process data store 154 may include an indication of a range or threshold level of spoilage organisms and weather conditions (e.g., rainfall, temperature, etc.) that may be used to determine the yield for a particular crop production process. Each of the indications of microbial measurements and/or non-microbial measurements that may be used to indicate the state (e.g., measure the success/failure) of each of the types of the processes may be known as a “risk vector” as described further below with respect to the risk assessment module 140.

In some embodiments, the microbial risk assessment system 100 may receive raw input data 110. The raw input data 110 may originate from one or more internal and/or external (e.g., third-party) computing systems. Each data point and/or data set included in the raw input data 110 may include one or more identifiers. As an example, the one or more identifiers may be included as metadata for each data point and/or data set included in the raw input data 110. In some cases, each data point and/or data set (e.g., temporal and/or spatial data set) included in the raw input data 110 may include at least one entity identifier and at least one process identifier. The entity identifier may be an identifier that corresponds to a particular entity that may be assessed by the microbial risk management system 100, where the entity identifier identifies an entity associated with the data point and/or data set. In some cases, the entity identifier may be associated with one or more geographic locations. For example, an entity identifier may be associated with one or more geographic locations corresponding to the entity's processes. The process identifier may be an identifier than corresponds to a particular type of process that may be correspond to a particular entity (e.g., managed by an entity). In some cases, a process identifier included with the raw input data 110 may provide contextual information for the data point and/or data set within the process to which the data point and/or data set corresponds (e.g., was derived). For example, for a data set of microbial data corresponding poultry yielded by a poultry production process managed by an entity, the data set may include process identifiers for a particular group of poultry, house (e.g., building) storing the group of poultry among a number of groups of poultry, location (e.g., facility) of the house, and time(s) and date(s) of the sampled measurements corresponding to the microbial data. The exemplary microbial risk assessment system 100 may be configured to account for differences in data sources and types corresponding to the raw input data 110. Given each data source's potentially unique insight of an entity and their respective processes, there can be two or more techniques used to take advantage of the respective data. Data source-specific modeling techniques may be applied to some or all of the data sources to demonstrate feasibility and validate the approach for each data source and modeling technique.

In some embodiments, the raw input data 110 may include microbial data 112, where the microbial data 112 corresponds to a particular process of an entity. The raw input data 110 may include non-microbial data 114, where the non-microbial data 114 corresponds to a particular process of an entity. Some non-limiting examples of microbial data 112 may include data collected from and/or using direct or indirect quantification, culture, sequencing, most-probable-number, secretion assays, polymerase chain reaction (PCR) and its variants, PIPER, VIPER, HYPER, immunoassays, whole genome sequencing, metagenomic sequencing, next generation sequencing, public and private data sources, historical lab reports, etc. As an example, for a process corresponding to poultry production, microbial data 112 may include measurements for microbes (e.g., microbial load, microbial growth cycling, identified serotype(s), etc.) related to Coccidia and/or Salmonella sampled from groups of poultry being raised in a production facility. Some non-limiting examples of non-microbial data 114 may include physical structures and accompanying descriptors (e.g. water sources, ventilation, lighting, humidity, temperature, bedding, age, condition, living dimensions, feeders, pens, etc.), temporal (e.g., seasonal, time of year, etc.) data, spatial (e.g., geographic, location, elevation, vegetation) data, business operations and financial data, imagery and remote sensing data, market pricing, process management practices, sanitation practices, pathogen control programs and treatments for outputs and/or environments corresponding to outputs, output-specific characteristic data for a process (e.g., final target output size, output growth rates, output mortality, breed/species/type of the output, etc.), production efficiency data (e.g., feed conversion rates, feed consumption, feed costs, average daily weight gain, total pounds of output produced, grower rank, mortality rate, cost per unit of live weight produced, etc.), and environmental (e.g., physical hazard, soil, weather, emissions and pollution) data. As an example, non-microbial data 114 may include weather and/or climate data for a geographic area or location (e.g., as defined by coordinates for a particular latitude and longitude) corresponding to an entity's process, where the weather and/or climate data may include indicators of natural disasters (e.g., hurricanes, flood, drought, tornados, etc.). The microbial data 112 and/or the non-microbial data 114 may be qualitative and/or quantitative. In some cases, the microbial data 112 and/or non-microbial data 114 may correspond to more than one process corresponding to more than one entity. For example, weather data for a particular geographic area may correspond to any suitable number of processes occurring within the geographic area that are managed by any suitable number of entities. In some cases, the microbial data 112 and non-microbial data included in the raw input data 110 may be historical data, such that the microbial risk assessment system 100 may determine a microbial risk assessment 170 and/or a loss calculation estimate 180 for particular entities at one or more instances in time (e.g., past, present, or future). For example, past historical data for a process included in the raw input data 110 may be used to determine entity's present or future risk for the process.

In some embodiments, data included in the microbial data 112 and/or the non-microbial data 114 may be sampled at one or more times during an individual process. Sampling times for the data and/or a number of samples collected may be based the type of the process from which the microbial data 112 is derived, the type of the microbial data 112 to be sampled, and/or the reproductive cycling of the microbe from which the microbial data 112 is derived. In some cases, sampling times may be selected to coincide within optimal (e.g., peak) detection periods within a microbial cycle corresponding to a process. In some cases, sampling times may be selected to coincide within optimal (e.g., peak) detection periods within a control and/or treatment program (e.g., pathogen control and/or treatment program) corresponding to a process. As an example, microbial data 112 may be collected and sampled based on a 4-7 day reproductive cycle (e.g., prepatent period) for parasites (e.g., Coccidia) in poultry.

In some embodiments, data included in the microbial data 112 and/or the non-microbial data 114 may be sampled from one or more geographic locations and/or subjects (outputs and/or products) corresponding to an individual process. Sampling locations and/or subjects for a particular process may be selected based on expediency and availability for sampling at the respective locations and/or subjects. In some cases, a subset of available geographic locations and/or subjects corresponding to a process may be sampled to obtain microbial data 112 and/or the non-microbial data 114. Such selective sampling may allow for extrapolation of microbial data 112 and/or non-microbial data to the process as a whole, while minimizing time and expenses for collecting microbial data 112 and/or non-microbial data 114. As an example, for a poultry production process corresponding to ten poultry facilities (e.g. farms), each facility with four houses containing distinct groups of poultry, microbial data 112 may be sampled from poultry in the distinct groups of poultry in two of the four houses on three of the ten farms. As an example of the above-described sampling techniques, for a process corresponding to poultry production, microbial data 112 including measurements for microbes related to Coccidia and/or Salmonella may be sampled from twelve poultry per house included in a production facility (e.g., poultry farm), where the production facility includes two houses, and three of an entity's production facilities are sampled according to the above criteria per week. Measurement for microbial data 112 and/or non-microbial data 114 may be sampled based on the status (e.g., maturation, reproductive cycle, microbial infection cycle) of the output of the process. For example, for the above described process for poultry production, measurements for microbes related to Coccidia and/or Salmonella may be sampled at days 17, 28, and 35 of the poultries' lifecycle (beginning from birth).

In some embodiments, the data aggregation module 120 may aggregate (e.g., receive) the one or more microbial data 112 and/or the non-microbial data 114. The data aggregation module 120 may aggregate the microbial data 112 and/or the non-microbial data 114 from one or more data sources, including internal and/or external computing systems. The data aggregation module 120 may periodically and/or continuously query the one or more data sources to acquire microbial data 112 and/or the non-microbial data 114. For example, the data aggregation module 120 may periodically query the one or more data sources for weather data corresponding to an geographic location(s) for an entity's processes. In some cases, the data aggregation module 120 may filter the received microbial data 112 and/or the non-microbial data 114. The data aggregation module 120 may filter missing and/or erroneous data included in the microbial data 112 and/or the non-microbial data 114. In some cases, at least some of the microbial data 112 and/or non-microbial data 114 may be used to provide an inference for an entity's process (or portion(s) of an entity's process) different from the process (or portion(s) of the entity's process) from which the microbial data 112 and/or non-microbial data 114 was derived as described herein. Based on aggregating the microbial data 112 and/or the non-microbial data 114, the data aggregation module 120 may provide aggregated data 116 to the data characterization module 130, where the aggregated data 116 includes at least some of the microbial data 112 and/or the non-microbial data 114.

In some embodiments, the data characterization module 130 may receive the aggregated data 116 from the data aggregation module 120. The data characterization module 130 may be communicatively coupled to the entity data store 152 and/or the process data store 154. Based on receiving aggregated data 116, the data characterization module 130 may analyze and characterize the aggregated data 116 (e.g., including at least some of the microbial data 112 and/or non-microbial data 114) as corresponding to one or more particular entities and one or more particular processes corresponding to the entity(ies). In some cases, the data characterization module 130 may identify an entity identifier included (e.g., as metadata) for each data point and/or data set of the aggregated data 116 and may compare the entity identifier to one or more entity identifiers included in the entity data store 152. Based on the comparison, the data characterization module 130 may identify the data point and/or data set as corresponding to the entity(ies) indicated by the entity identifier(s). In some cases, the data characterization module 130 may identify one or more process identifiers included (e.g., as metadata) for each data point and/or data set and may compare the process identifier(s) to one or more process identifiers included in the process data store 152. Based on the comparison, the data characterization module 130 may identify the data point and/or data set as corresponding to a type of process indicated by the process identifier. One or more processes may correspond to a particular entity, such that one or more data (e.g., microbial data 112 and/or non-microbial data 114) corresponding to one or more processes may be attributed to a particular entity. For example, data corresponding to microbial measurements originating from a group of poultry being raised at a production facility may be attributed to a particular poultry production entity, as well as to a holding entity of which the poultry production entity is a subsidiary entity. In some cases, a data point and/or data set included in the aggregated data 116 may correspond to more than one process as described herein. In some cases, the data characterization module 130 may sample data from aggregated data 116 (e.g., including the microbial data 112 and/or the non-microbial data 114) based on the sampling techniques described herein (e.g., temporal sampling, geospatial sampling, and/or subject-based sampling), such that the sampled data from the microbial data 112 and/or non-microbial data 114 is included in the characterized data 118 provided to the risk assessment module 140. Based on analyzing and characterizing the aggregated data 116, the data characterization module 130 may provide characterized data 118 to the risk assessment module 140.

In some embodiments, the risk assessment module 140 may receive characterized data 118 from the data characterization module 130. The risk assessment module 140 may be communicatively coupled to the entity data store 152 and/or the process data store 154. The characterized data 118 may include one or more data points and/or data sets, where each data point and/or data set is characterized as microbial data (e.g., microbial data 112) or non-microbial data (e.g., non-microbial data 114). Each data point and/or data set may be further characterized as corresponding to one or more particular entities (e.g., as indicated by an entity identifier) and corresponding to a particular type of process (e.g., as indicated by a process identifier). In some cases, data points and/or data sets included in the characterized data 118 may include additional process identifier(s) providing contextual information for the data within the respective process(es) to which the data corresponds. Accordingly, the risk assessment module 140 may determine and/or otherwise identify entity-specific data (e.g., originating from the raw input data 110) that corresponds to each entity and each of the processes (e.g., including the types of the process(es)) corresponding to the respective entity. Based on the entity-specific data corresponding to each entity, the risk assessment module 140 may determine one or more risk vector ratings (e.g., numerical ratings, grades, and/or other state indicators) corresponding to one or more of (e.g., each of) the entity's processes, which may be used to determine and/or may be included in a microbial risk assessment 170 for each respective entity. In some cases, the risk assessment module 140 may only use data (e.g., included in the characterized data 118) corresponding to a particular process for an entity to determine risk vector ratings for that process of the entity. In other cases, the risk assessment module 140 may use data (e.g., included in the characterized data 118) corresponding to processes of an entity's affiliates to determine risk vector ratings for that process of the entity. In some cases, the risk assessment module 140 may using one or more microbial risk assessments 170 to determine (e.g., calculate) one or more loss calculation estimates 180. Each microbial risk assessment 170 may be used individually or in combination with others to calculate a particular loss calculation estimate 180 for an entity, which may be used as a basis for one or more financial models and/or products such as insurance policies, derivatives, etc.

In some embodiments, to determine a microbial risk assessment 170 for process(es) corresponding to a particular entity, the risk assessment module 140 may determine one or more risk vector ratings that correspond to the respective data (e.g., microbial data 112 and/or non-microbial data 114) for one or more of (e.g., each of) the entity's processes. Risk vector ratings may be determined for a risk vector based on one or more metrics (e.g., including a measurement, value, number, or amount) and/or one or more evaluations (e.g., including a categorical or binary determination) associated with the risk vector, where the metric(s) or evaluation(s) are derived from at least some of the characterized data 118 that is indicative of a particular risk vector. Each process (e.g., as stored in the process data store 154) may be mapped to one or more risk vectors are indicative of a state of the process. As an example, risk vectors for a production of a particular crop (e.g., corn, soy, wheat, etc.) may correspond to microbial levels in crops' soil and weather data corresponding to the crops' geographic location. In some cases, one or more risk vectors may be related (e.g., correlated). As an example, spoilage organism microbial levels for a particular crop may be correlated with temperature, sunlight, and/or rainfall levels for the geographic area in which the crop is located.

In some embodiments, a risk vector rating may be equivalent to a metric or evaluation derived from characterized data 118. In some cases, a risk vector rating may be determined based on providing the one or more metrics and/or evaluations for a risk vector as an input to a model (e.g., a partially pooled Bayesian model or a categorical assessment model) for the risk vector. In some cases, providing a metric as an input to the model may include mapping the metric to one or more thresholds or ranges that are indicative of a risk vector rating. In some cases, providing a metric as an input to the model may include applying one or more transformations (e.g., statistical analyses, normalizations, weightings etc.) to the metric to determine a risk vector rating. As an example, the one or more transformations may include statistical analysis techniques, normalization techniques (e.g., that are based on an expected statistical distribution), and/or weighting techniques. In some cases, providing the evaluation as an input to the model may include mapping the evaluation to one or more categories or partitions that are indicative of a risk vector rating. In some cases, the risk vector rating for a risk vector may be a function of two or more metrics and/or evaluations corresponding to the risk vector. As an example, risk vector rating may corresponding to a value for a ratio of a feed conversion rate and a microbial load value. In some cases, determining a risk vector rating for a risk vector may include normalizing a metric and/or comparing the metric to a particular expected statistical distribution (e.g., binomial distribution, normal distribution, beta distribution, etc.) corresponding to the metric.

In some embodiments, the risk assessment module 140 may generate a time-series of risk vector ratings for risk vectors based on temporal (e.g., time-series) data included in the characterized data 118. Each risk vector rating included in the time-series of risk vector ratings may provide an indication of a state of a risk vector at the time to which the respective risk vector rating corresponds. A risk vector rating included in a time-series of risk vector ratings may be determined based on a metric and/or evaluation determined using characterized data available at the time to which the respective risk vector rating corresponds. By generating time-series of risk vector ratings, an entity and the entity's affiliate may review and evaluate changes in the risk vector ratings over a window of time (e.g., 1 month, 6 month, 1 year, 2 years, etc.) corresponding to the time-series. Further, the risk assessment module 140 may determine and track metrics corresponding to risk vector ratings, such as average risk vector rating, complex average risk vector rating, median risk vector rating, minimum risk vector rating, maximum risk vector rating, etc. By receiving and processing updated raw input data 110 using the microbial risk assessment system 100 as described herein, additional risk vector ratings may be determined for an entity's process(es).

In some embodiments, the risk assessment module 140 may determine a risk vector rating for a risk vector based on (e.g., as a function of) a previous risk vector rating. The risk assessment module 140 may determine a risk vector rating based on at least one previous risk vector rating based on a relationship between the present and previous risk vector ratings. In some cases, the relationship may be a temporal relationship and/or a spatial relationship. As an example, for a temporal relationship, a risk vector rating for microbial load at a particular production facility for poultry production may be at least partially determined as a function of previous risk vector rating for the microbial load at the same production facility at a previous time (e.g., 1 year in the past). As another example, for a spatial relationship, a risk vector rating for microbial load at a first house of particular production facility for poultry production may be at least partially determined as a function of previous risk vector rating for the microbial load at a second house that was geographically located within the proximity of the first house at the same production facility. To determine a risk vector rating for a risk vector based on a previous risk vector rating, the risk assessment module 140 may use first characterized data 118 used to determine the previous risk vector rating in combination with second, new characterized data 118 that was not used to determine the previous risk vector rating.

In some embodiments, as described herein, a risk vector may be based on observable information (e.g., characterized data 118) for an entity that is indicative of a state of an entity's (e.g., affiliate's) process. This observable information can be categorized into observable subject areas (e.g., risk vectors), which can each be independently determined and/or characterized. For example, one possible proxy for the health of an entity's livestock herd may be infectious diseases rates for the herd (e.g., as indicated by microbial data 112). Some non-limiting examples of risk vectors for processes may include:
microbial load, microbial composition profile, microbial species, microbial subtypes, microbial serotypes, and/or microbial strains;
rate of change of a microbial population (e.g., growth, cycling, etc.) during a process;
co-morbidities and other concurrent health effects;
output-specific characteristics (e.g. product-specific characteristics) for a process;
efficacy of control programs and treatments (vaccines, chemicals, probiotics, etc.) to the process;
environmental conditions (e.g., weather, physical structures, available natural resources);
feeding conditions;
production efficiencies (e.g., feed conversion, feed costs, average daily gain, etc.);
house profiles;
geographic location;
spatial data within a geographic location (e.g., location of a group of poultry within a particular house within a production facility)
historical location performance, including: intervention usage at site (e.g. chemical, biological, physical, etc.), personnel, site, and house records;
financial (e.g., market) and economic conditions;
process management practices; and
microbial risk profile(s) (e.g., microbial risk rating(s)) of an entity's affiliate(s).

As an example, for a process corresponding to poultry production, characterized data 118 may include temporal measurements for microbes related to Coccidia from poultry at multiple farms owned by an entity, where microbial load for microbes related to Coccidia functions as a risk vector for the poultry production process. Characterized data 118 including measurements for the microbes may be sampled at a number of instances during the lifecycle of the poultry as described herein, where the measurements are expected to have an assumed distribution of a negative binomial distribution. As described herein, to determine risk vector ratings, the risk assessment module 140 may apply one or more transformations to metrics corresponding to characterized data 118. In the example, the risk assessment module 140 may assign a Coccidia curve score to the respective group of microbial data for each of the sampled farms, where the microbial data is plotted as a time-series and where the Coccidia curve score indicates a level of Coccidia microbes present among the groups of poultry of the sampled farms. The risk assessment module 140 may calculate a difference score for each group of microbial data as compared to each of the other groups of microbial data corresponding to the farms. The risk assessment module 140 may convert the difference score for each group of microbial data to a similarity score. The risk assessment module 140 may multiply the respective similarity score by the assigned Coccidia score for each group of microbial data. The risk assessment module 140 may calculate a weighted Coccidia score for each group of microbial data, where the weighted Coccidia score functions as the risk vector rating for the risk vector for microbial load for microbes related to Coccidia. The weighted Coccidia score may be used by the risk assessment module 140 to determine a risk vector score for the poultry production process as described herein (e.g., by inputting to the weighted Coccidia score to a partially pooled Bayesian model).

As another example, for a process corresponding to poultry production, characterized data 118 may include temporal measurements for microbes related to Coccidia from poultry at multiple farms owned by an entity, where microbial load for microbes related to Coccidia functions as a risk vector for the poultry production process. Characterized data 118 including measurements for the microbes may be sampled at a number of instances during the lifecycle of the poultry as described herein and the measurements may be recorded as temporal data for the lifecycle of the poultry (e.g., a “growout” period). Such temporal data may function as a risk vector for microbial load for microbes related to Coccidia and the risk assessment module 140 may determine a risk vector rating based on the temporal data. Additional risk vectors and risk vector ratings for production metrics relating to the poultry (e.g., feed conversion ratio, average daily weight gain, mortality, weight, and/or feed costs) may be derived from the characterized data. The risk assessment module 140 may determine a risk vector score for the poultry production process as described herein based on a weighted combination of the risk vector ratings for the temporal microbial load data and production metrics. Further, cycling patterns indicated by the temporal microbial load data and production metrics (e.g., available via a microbial risk assessment 170) may be used by an entity associated with the poultry production process to identify the efficacy of a control and treatment program for Coccidia.

As another example, for a process corresponding to poultry production, characterized data 118 may include temporal measurements for microbes related to Salmonella from poultry at multiple farms owned by an entity, where identified serotype(s) for microbes related to Salmonella functions as a risk vector for a prevalence of Salmonella in the poultry production process. Characterized data 118 including measurements for the microbes and identified serotype(s) may be sampled at a number of instances during the lifecycle of the poultry as described herein and the measurements may be recorded as temporal data for the lifecycle of the poultry (e.g., a “growout” period). The measurements for the identified serotype(s) may function as a risk vector for a prevalence of Salmonella and the risk assessment module 140 may determine a risk vector rating based on the temporal data. To determine the risk vector rating, the risk assessment module 140 may input the identified serotype(s) to a categorical assessment model including a number of risk tiers, each risk tier corresponding a particular risk vector rating. Each of the risk tiers (e.g., two or more risk tiers) may correspond to a particular level of risk and related risk vector rating to the poultry production process. In some cases, a first serotype for Salmonella may exhibit a greater risk to the poultry production process, while a second serotype for Salmonella may exhibit a reduced risk to the poultry production process relative to the first serotype. The risk assessment module 140 may determine a weighted risk vector rating for the prevalence of Salmonella based on the categorical assessment model and the risk vector ratings mapped to the risk tiers for identified serotypes of Salmonella, where the weighted risk vector rating is determined based on risk tiers corresponding to identified serotypes and the prevalence of the identified serotypes (e.g., the frequency and/or or number of times a particular serotype was identified. The risk assessment module 140 may determine a risk vector score for the poultry production process as described herein based on a weighted combination of the risk vector ratings for the prevalence of Salmonella and one or more production metrics, where production metrics can include remaining reproductive time (e.g., for breeding poultry) and total pounds of poultry at risk of Salmonella contamination (e.g., for meat produced poultry). In some cases, the risk assessment module 140 may determine such risk vector ratings and the risk vector score for hierarchical levels of a process that are lower than the process level, such as for individual group(s) of poultry from which characterized data 118 is derived and individual houses and/or production facilities that house groups of poultry for the poultry production process. In some cases, the risk assessment module 140 may determine a time-series of risk vector scores for the poultry production process over time based on changes to the risk vector ratings for the prevalence of Salmonella and the one or more production metrics, thereby enabling an entity to identify temporal and spatial risks for individual groups of poultry over time. Such a time-series of risk vector scores can be used by the risk assessment module 140 to determine a time series of microbial risk ratings as described herein.

In some embodiments, as described herein, risk vectors may be mapped to one or more ratings, where each rating corresponds to a quantitative or qualitative indication of a state (e.g., poor, satisfactory, good, excellent) of a risk vector. The risk assessment module 140 may determine and/or store the mappings of ratings to risk vectors for an entity's process(es) and/or each of an entity's affiliate's process(es), where the ratings for risk vectors are determined based on the characterized data 118. In some cases, based on the ratings for each of the risk vectors for an entity's process, the risk assessment module 140 may determine a risk vector score. The risk vector score may be determined based on a combination of the ratings for each of the risk vectors for the entity's process. In some cases, the risk vector score may be determined based on providing the risk vector ratings for the process as an input to a model (e.g., a partially pooled Bayesian model or a categorical assessment model), where the model applies one or more predictive modeling techniques and/or transformations (e.g., statistical analyses, normalizations, and/or weightings) to the received risk vector ratings. In some cases, a model may receive one or more expected statistical distributions for characterized data 118 used to determine risk vector ratings, where the expected statistical distributions are used to determine the risk vector score. For example, when the risk assessment module 140 uses a partially pooled Bayesian model to determine a risk vector score, the risk assessment module 140 may receive expected statistical distributions (e.g., normal distribution, negative binomial distribution, etc.) for microbial measurements used to determine risk vector ratings for a process and may use the statistical distributions when determining the risk vector score.

In some cases, the risk assessment module 140 may use a number of models to determine risk vector ratings and a risk vector score for a process as described herein. In some cases, the risk assessment module 140 may use a model that applies transformations to combinations of risk vector ratings. For example, the risk assessment module 140 may divide a feed conversion rate for farms raising groups of poultry by a weighted Coccidia scores for Coccidia microbes determined for the farms to produce a quantitative assessment of the impact of the Coccidia microbe on the feed conversion rates for the poultry. In some cases, a model for determining a risk vector score for a process may receive risk vector ratings corresponding to risk vectors derived from microbial data 112 and/or non-microbial data. As an example, a partially pooled Bayesian model may receive risk vector ratings for weather, location, and historical performance data along with risk vector ratings for a time-series of measured Coccidia loads to determine a risk vector score for an adjusted feed conversion ratio for a poultry production process.

In some embodiments, particular risk vectors may be associated with a weight (e.g., numerical weight), such that a weighted risk vector score may be determined from the ratings associated with the one or more risk vectors for the particular process. In some cases, the risk vector score may be weighted based on the timing of sampling of data (e.g., microbial data 112 and/or non-microbial data 114) during a particular process, where the data is included in the characterized data 118. Weights may be applied to risk vector ratings based on the time(s) during a particular process at which data was sampled for ingestion to microbial risk assessment system 100. As an example, first microbial data 112 indicating the presence of harmful pathogens that is collected at a first, earlier time in a process for poultry production may be weighted higher (e.g., for increased risk) than second microbial data 112 indicating the presence of harmful pathogens that is collected at a second, later time in the process for poultry production.

In some embodiments, the risk assessment module 140 may generate a time-series of risk vector scores for a process based on a time-series of risk vector ratings. Each risk vector score included in the time-series of risk vector scores may provide an indication of a microbial state of a process at the time to which the respective risk vector score corresponds. A risk vector score included in a time-series of risk vector scores may be determined based on risk vector ratings as described wherein, wherein the risk vector ratings are those available at the time to which the respective risk vector score corresponds. By generating time-series of risk vector scores, an entity and the entity's affiliate may review and evaluate changes in the risk vector scores for a process over a window of time (e.g., 1 month, 6 month, 1 year, 2 years, etc.) corresponding to the time-series. Further, the risk assessment module 140 may determine and track metrics corresponding to risk vector scores, such as average risk vector score, complex average risk vector score, median risk vector score, minimum risk vector score, maximum risk vector score, cumulative risk vector score, etc. By receiving and processing updated raw input data 110 using the microbial risk assessment system 100 as described herein, additional risk vector scores may be determined for an entity's process.

In some embodiments, one or more processes corresponding to an entity may be associated with a weight (e.g., a numerical weight), such that a microbial risk rating may be determined from a non-weighted or weighted combination of the risk vector scores for the one or more processes. If only one process corresponds to particular entity, the entity's microbial risk rating may be determined based on a respective risk vector score (e.g., non-weighted risk vector score or a weighted risk vector score) for the process as described herein. If more than one process corresponds to a particular entity, the entity's microbial risk rating may be determined based on a combination (e.g., weighted combination) of the respective non-weighted risk vector scores or weighted risk vector scores for one or more of (e.g., each of) the entity's processes as described herein.

In some embodiments, each of the determined risk vector ratings and risk vector scores for an entity may be included and made available for evaluation in a generated microbial risk assessment 170 for a particular entity. The microbial risk assessment 170 may include an indication (e.g. a written description, visual score card, and/or a graphical display) of the processes corresponding to the entity for which the microbial risk assessment 170 was generated and each of the risk vector ratings and risk vector scores for the respective processes. In some cases, the microbial risk assessment 170 may include the respective risk vector score or weighted risk vector score for each of the processes corresponding to the entity for which the microbial risk assessment 170 was generated. In some cases, indications of transformations, models, and/or other processing techniques used to derive risk vector ratings, risk vector scores, and microbial risk ratings may be included in a microbial risk assessment 170. For an entity corresponding to only one process, the microbial risk assessment 170 may include the respective risk vector score or weighted risk vector score as the microbial risk rating. For an entity corresponding to more than one process, the microbial risk assessment 170 may include a microbial risk rating determined as a combination (e.g., weighted combination) of the non-weighted risk vector scores or weighted risk vector scores for the entity's processes. In some cases, the risk assessment module 140 may determine the microbial risk rating by applying one or more transformations to risk vector score(s) corresponding to an entity. The microbial risk assessment 170 and an included microbial risk rating may be indicative of the entity's microbial risk profile, including the entity's past, present, and/or future risk of adverse events and/or outcomes for their respective process(es). For example, the microbial risk assessment 170 may be indicative of risk to yield of the entity's processes.

In some embodiments, the risk assessment module 140 may generate a time-series of microbial risk ratings for an entity based on a time-series of risk vector scores for at least one of entity's processes. Each microbial risk rating included in the time-series of microbial risk ratings may provide an indication of a microbial state of a number (e.g., all) of an entity's processes at the time to which the respective microbial risk rating corresponds. A microbial risk rating included in a time-series of microbial risk ratings may be determined based on risk vector scores as described wherein, wherein the risk vector scores are those available at the time to which the respective microbial risk rating corresponds. By generating a time-series of microbial risk ratings, an entity and the entity's affiliate may review and evaluate changes in the microbial risk rating for an entity over a window of time (e.g., 1 month, 6 month, 1 year, 2 years, etc.) corresponding to the time-series. Further, the risk assessment module 140 may determine and track metrics corresponding to a microbial risk rating for an entity, such as average microbial risk rating, complex average microbial risk rating, median microbial risk rating, minimum microbial risk rating, maximum microbial risk rating, cumulative microbial risk rating, etc. By receiving and processing updated raw input data 110 using the microbial risk assessment system 100 as described herein, additional microbial risk ratings may be determined for an entity.

In some embodiments, the risk assessment module 140 (and microbial risk assessment system 100) may output one or more microbial risk assessments 170. Each microbial risk assessment 170 may belong and/or otherwise correspond to a respective entity via being at least partially derived based on raw input data 110 corresponding to the entity. Based on a microbial risk assessment 170 including risk vector ratings and scores corresponding to a number of the entity's processes, an entity and/or an entity's affiliate may analyze the microbial risk assessment 170 and may identify potential areas of risk corresponding to the entity's processes. In some cases, using a microbial risk assessment 170, an entity or entity's affiliates may determine a degree to which individual risk vectors positively and/or negatively impacted the microbial risk assessment 170. To mitigate potential areas of risk, in some cases, an entity may introduce and/or otherwise apply one or more risk mitigation measures (e.g., pathogen control and/or treatment programs) to their respective processes to reduce the risk associated with risk vectors that negatively impact their microbial risk profile as indicated by the microbial risk assessment 170. As an example, for a higher risk rating associated with a risk vector for bacterial disease in a livestock herd, an entity may provide the livestock herd with antibiotic treatments and may observe changes to the risk vector rating corresponding to a presence of the bacterial disease for a process corresponding to producing the livestock herd. Further, an entity may identify process management techniques that positively and/or negatively impact their microbial risk profile as indicated by the microbial risk assessment 170. As an example, a lower risk rating associated with a risk vector for bacterial disease in a particular farm (e.g., determined based on the farm having desirable microbial characteristics) can indicate that process management practices are effective and may be applied to other farms having higher risk ratings associated with the risk vector for bacterial disease. As another example, a change from a higher risk rating to associated with a risk vector for bacterial disease in a particular farm (e.g., determined based on the farm having desirable microbial characteristics) to a lower risk rating can indicate that an effectiveness of a control and treatment program for the bacterial disease has reduced over time.

In some embodiments, the risk assessment module 140 (and the microbial risk assessment system 100) may output one or more loss calculation estimates 180. Each loss calculation estimate 180 may belong and/or otherwise correspond to a respective entity. In some cases, a loss calculation estimate 180 may be an indicator (e.g., numerical indicator) of an expected loss associated with an individual process or a combination of processes corresponding to an entity. Examples of expected loss may include expected economic loss, financial loss, or output (e.g., product) loss associated with an individual process or a combination of processes corresponding to an entity. In some cases, the expected loss may be associated with an expected loss for an output of a process. The risk assessment module 140 may determine (e.g., calculate) the loss calculation estimates 180 using one or more loss calculation models and/or statistical methods. Example loss calculation models and/or statistical analysis techniques used by the risk assessment module 140 can include modeling of the performance of process operations (e.g. inputs corresponding to process outputs) and losses from impacts associated with risk vector rating and/or score data using unaided learning methods, Bayesian and/or non-Bayesian predictive functions, and/or other artificial intelligence (e.g., machine learning and neural network) techniques. In some cases, models and methods utilized to determine the health effects and performance impact on process operations may be applied at various levels throughout hierarchical levels corresponding to a process of an entity (e.g., an output product of a process, a group of output products of the process, geographic units including the output product, houses, farms, facilities, complexes, company, organization, etc.).

In some cases, one or more microbial risk assessments 170 may be provided as inputs to determine the loss calculation estimates 180. For example, risk vector ratings and/or scores corresponding to an entity's microbial risk assessment 170 may be used as input parameters in a loss calculation model to determine a loss calculation estimate 180.

In some embodiments, a microbial risk assessment 170 and/or a loss calculation estimate 180 for an entity may be provided to an entity's affiliates (and one or more other entities), such that the affiliates may analyze the entity's microbial risk assessment 170 and/or loss calculation estimate 180. Using the entity's microbial risk assessment 170 and/or loss calculation estimate 180, affiliates may evaluate a state of their business relationships with the entity. For example, an affiliate (e.g., food production entity) maintaining a supply chain relationship with an entity (e.g., a food producer) may need to reevaluate their supply chain relationship based on determining the entity to have a poor and/or otherwise unsatisfactory microbial risk assessment 170 for a production process. In some cases, one or more third-party entities may receive a microbial risk assessment 170 and/or a loss calculation estimate 180 for a particular entity. Third-party entities may include one or more entities associated with the financial industry, including (but not limited to) insurance, lending, re-insurance, banking, and underwriting entities. The third-party entities may receive one or more microbial risk assessments 170 and/or loss calculation estimates 180 corresponding any number of entities, such that the third-party entities may use the microbial risk assessments 170 and/or loss calculation estimates 180 to derive one or more financial products for entity, an entity's affiliate, or any other suitable entity or individual. As described herein, some non-limiting examples of financial products may include risk transfer products, credit products, derivative products, traditional and/or non-traditional business (or product) valuation products, securitization products, etc. As an example, using a microbial risk assessment 170 and/or a loss calculation estimate 180, an insurance entity may determine a loss prediction (e.g., financial loss, product loss, etc.) for an output (e.g., output product) associated with a particular process and may provide risk transfer products to insurance a risk corresponding to the output.

In some embodiments, third-party entities may use a particular (or one or more) microbial risk assessment(s) 170 and/or a loss calculation estimate 180 as an input for models to create financial products. In some cases, underlying microbial data 112, non-microbial data 114, risk vector ratings and scores included in a microbial risk assessment 170, and/or loss calculation(s) included in a loss calculation estimate 180 may be used as an input for models to create financial products. In some cases, microbial risk assessments 170 may be used to assess the risk of adverse events for processes. Accordingly, financial (e.g., insurance) products may be created an entity and/or an entity's affiliates, where the financial products correspond to the risk of adverse events for a particular process corresponding to the entity. Such financial products may allow a third-party entity (e.g., financial entity) to pool risk for the adverse events and create new types of financial products corresponding to one or more processes for an entity and its affiliates. As an example, third-party entities (e.g., financial entities) may use microbial risk assessments 170 and/or a loss calculation estimates 180 to create and/or derive financial products for intervention entities (e.g., affiliates) having direct and/or indirect business relationships with an entity, including chemical production entities, vaccine production entities, sanitation entities, feed additive entities, manufacturing machinery entities, logistics service provider entities, product manufacturer and/or provider entities, etc. As another example, third-party entities (e.g., financial entities) may use microbial risk assessments 170 and/or loss calculation estimates 180 to create and/or derive financial products for other financial entities service entities (e.g., affiliates) having direct and/or indirect business relationships with an entity, including entities such as other insurance broker entities, captive insurance provider entities, insurance entities, securities and/or valuation services providers, and underwriter entities.

In some embodiments, the microbial risk assessment system 100 may be implemented as software executed on one or more computer systems 300. For example, the microbial risk assessment system 100 may be implemented as software executed on an entity's computer systems or a third-party entity's computer systems, where the third-party entity (e.g., risk management service provider) provides services to the entity. In some embodiments, the microbial risk assessment system 100 may provide a user interface (not shown in FIG. 1). In some cases, the user interface may be accessible via a web-based application (e.g., available at a website accessible via a web browser) and/or a mobile application (e.g., installed at or operating on a mobile computing device). The user interface may present (e.g., display) information regarding the microbial risk assessments 170 and loss calculation estimates 180 of an entity and/or an entity's affiliates. For example, the user interface may present (e.g., display) information regarding the microbial risk profile of an entity and/or the entity's affiliates. In some cases, written reports indicative of information included in the microbial risk assessments 170 and loss calculation estimates 180 may be available at and/or provided by the user interface. In some embodiments, the user interface may provide interactive components whereby a user may interact with the microbial risk assessment system 100. For example, by interacting with a user interface, the user may view microbial risk assessments 170 and/or loss calculation estimates 180 corresponding to their respective entity and/or their entity's affiliates. In some cases, the user interface may provide interactive components whereby a user may interact with the data aggregation module 120 and/or the data characterization module 130. For example, by interacting with a user interface, the user may review data source for the raw input data 110 and may filter, interpolate, and/or otherwise modify raw input data 110 that is ingested into the microbial risk assessment system 100.

In some embodiments, the microbial risk assessment system 100 may make microbial risk assessments 170 and/or loss calculation estimates 180 of an entity and/or an entity's affiliates available via a network (e.g., internet, intranet, cellular network, etc.). For example, the microbial risk assessment system 100 may send and/or cause sending of microbial risk assessments 170 and/or loss calculation estimates 180 to a recipient (e.g., an individual corresponding to an entity or entity's affiliates) via Short Messaging Service (SMS), email, internet-based messaging, and/or social media messaging techniques. In some cases, the microbial risk assessment system 100 may make microbial risk assessments 170 and/or loss calculation estimates 180 available via a mobile application or internet browser application operating on a computing device (e.g., mobile computing device).

In some embodiments, the user interface may provide interactive components whereby a user may interact with the risk assessment module 140. For example, by interacting with a user interface, the user may review ratings, scores and/or weightings mapped to risk vectors for the entity's processes and each of the processes corresponding to the entity's affiliates. In some cases, a user may configure risk vectors corresponding to biological and other processes, such that a user may configure the mapping of microbial data 112 and/or non-microbial data 114 to risk vectors that impact the outcome of a particular process and/or indicate a risk to the particular process. A user may configure ratings and/or scores corresponding to risk vectors, such that the user can configure ranges, thresholds, and/or categories (e.g., bins) used to determine risk vector ratings for risk vectors and risk vector scores for processes. The risk assessment module 140 may also calculate loss calculation estimates 180 based on the ratings and risk vectors included in one or more microbial risk assessments 170. In some cases, a user may configure types and/or factors of loss calculation models (e.g., equations) used to determine the loss calculation estimates 180.

### Some Embodiments of Microbial Risk Assessment Method

In some embodiments, as described herein, microbial data (e.g., microbial data 112) and non-microbial data (e.g., non-microbial data 114) corresponding to a particular process may be used to assess an entity's risk and/or a risk for each of the entity's affiliates for the process. Accordingly, the microbial risk assessment system 100 as described herein may execute a method (e.g., assessment method) to determine one or more microbial risk assessments for processes corresponding to any suitable number of entities. The method may be used to determine a microbial risk assessment (e.g., microbial risk assessment 170) for a particular entity, where the microbial risk assessment corresponds to one or more processes managed and/or otherwise associated with the entity. The method may be performed periodically, such that a microbial risk assessment for an entity is periodically determined (e.g., updated or refreshed) to account for new input data (e.g., microbial and/or non-microbial) data corresponding to the entity's ongoing and/or previous processes. To determine a microbial risk assessment for an entity, the microbial risk assessment system 100 (or any other suitable computing system) may execute the assessment method by (1) aggregating one or more microbial and non-microbial data from one or more data sources; (2) identifying and/or characterizing (e.g., based on one or more entity identifiers and one or more process identifiers) the aggregated data as corresponding to the least one biological process of at least one entity; (4) mapping the characterized data to one or more risk vectors corresponding to the at least one biological process; (4) determining, based on the characterized data, one or more risk vector ratings for the one or more risk vectors corresponding to the at least one process of the at least one entity; (5) determining, based on the one or more risk vector ratings for the at least one process, a microbial risk assessment for the entity; and (6) determining, based on the microbial risk assessment, an expected loss value (e.g., as a part of a loss calculation estimate) for the at least one entity. The assessment method may further include (7) providing and/or otherwise causing sending of the microbial risk assessment and/or the expected loss value to the entity and/or one or more affiliates of the entity.

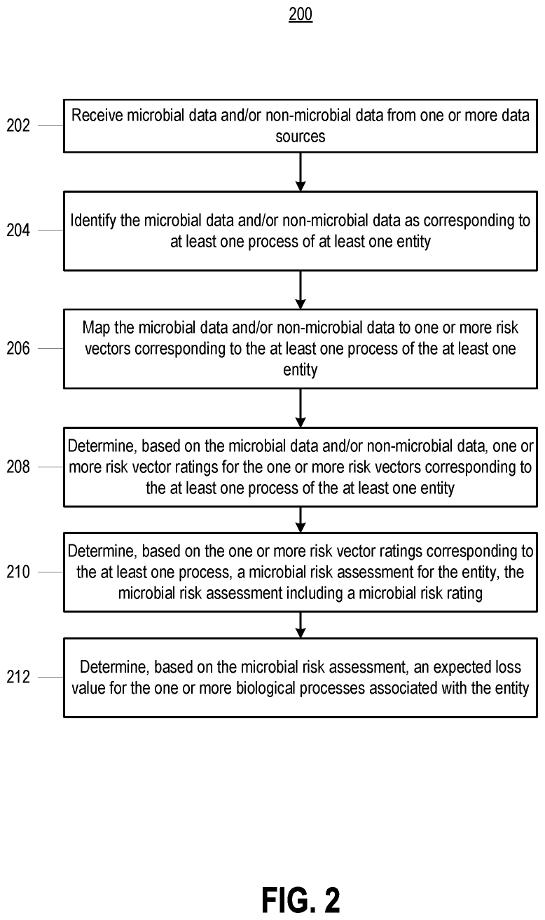

FIG. 2 shows a flowchart of an entity-specific microbial risk assessment method 200, according to some embodiments. The method 200 may be suitable for determining one or more microbial risk assessments and/or loss calculation estimates, where each microbial risk assessment and loss calculation estimate corresponds to the process(es) managed and/or otherwise associated with a particular entity. The resulting microbial risk assessments (e.g., microbial risk assessments 170) and/or loss calculation estimates (e.g., loss calculation estimates 180) may be provided to an entity, and entity's affiliates, and one or more third-party entities for further analyses as described herein, including risk assessment and derivation of one or more financial products. In some cases, the microbial risk assessments may include additional information as described herein, such as risk vector ratings for risk vectors, risk vector scores for processes, and rating methodology used to determine the risk vector ratings, risk rating scores, and microbial risk rating included in the microbial risk assessment.

For simplicity, the following paragraphs describe steps 202-212 of the method 200 with reference to determining a single microbial risk assessment and a single expected loss value for a single entity. However, one of ordinary skill in the art will appreciate that steps 202-212 may be performed in parallel and/or repeated for any suitable number of processes corresponding to any suitable number of entities, such that more than one microbial risk assessment and/or expected loss value may be determined.

At step 202, the microbial risk assessment system 100 may receive and/or aggregate one or more microbial data (e.g., microbial data 112) and/or non-microbial data (e.g., non-microbial data 114) from one or more data sources. Data sources may include microbial and non-microbial data provided and/or collected from one or more internal and/or external (e.g., third-party) computing systems as described herein with respect to FIG. 1. In some cases, the data aggregation module 120 may aggregate the microbial data 112 and/or the non-microbial data 114 as raw input data 110. Each of the data points and/or data sets included in raw input data 110 may include at least one entity identifier and at least one process identifier, such that each data point and/or data set may be characterized as corresponding to one or more particular entities and processes. The data aggregation module 120 may periodically and/or continuously aggregate (e.g., query) the microbial data 112 and/or the non-microbial data 114 from the one or more data sources. In some cases, the one or more data sources may provide the microbial data 112 and/or the non-microbial data 114 to the data aggregation module 120 (and the microbial risk assessment system 100 as a whole). Based on a configuration of the data aggregation module 120, in some cases, the data aggregation module may filter the aggregated microbial data 112 and/or the non-microbial data 114 as described herein. For example, the data aggregation module 120 may filter microbial data 112 that includes missing and/or erroneous data values. The data aggregation module 120 may provide aggregated data 116 to the data characterization module 130.

In some embodiments, the microbial data (e.g., received at step 202) may include data collected from at least one of: direct or indirect quantification, culture, sequencing, most-probable-number, secretion assays, polymerase chain reaction (PCR), PIPER, immunoassays, whole genome sequencing, metagenomic sequencing, and next generation sequencing techniques. As described herein, at least some of the microbial data corresponding to a particular biological process may be sampled at one or more times (e.g., a first time and a second time) based on a microbial lifecycle (e.g., of at least one microbe) corresponding to one or more biological processes. In some cases, the microbial data may include, for example, microbial load data for Coccidia and/or microbial serotype data for Salmonella.

In some embodiments, the non-microbial data (e.g., received at step 202) may include at least one of: physical structure data for the one or more biological processes, temporal data, spatial data, business operations and financial data, imagery and remote sensing data, economic data, management practices for one or more biological processes, sanitation practices for one or more biological processes, pathogen control program data for one or more biological processes, data corresponding to an output of the one or more biological processes, efficiency data for one or more biological processes, and environmental data for one or more biological processes. The one or more biological processes to which the microbial data and/or non-microbial data corresponds may include, for example, at least one of: a food production supply chain process, a crop production process, an animal production process, a pharmaceutical production process, a chemical manufacturing process, a feed production process, and a medical device manufacturing process. In some cases, the animal production process may be a poultry production (e.g., chicken growth) process.

At step 204, the microbial risk assessment system 100 may characterize and/or otherwise identify the microbial data 112 and/or the non-microbial data 114 as corresponding to at least one process of at least one entity based on one or more entity identifiers and one or more process identifiers included in the aggregated data (e.g., as metadata). The data aggregated at step 202 may be provided to the data characterization module 130 as aggregated data 116. The data characterization module 130 may characterize the aggregated data 116 determined by the data aggregation module 120 at step 202. As described herein, each data point and/or data set included in the aggregated data 116 may correspond to at least one entity identifier and at least one process identifier, such that the aggregated data 116 may be segmented as corresponding to one or more processes of one or more entities. If a data point and/or data set included in the aggregated data 116 does not include an entity identifier and/or a process identifier, the data characterization module may automatically assign the data an entity identifier and/or a process identifier based on the one or more entity and process identifiers included in the entity data store 152 and the process data store 154, respectively. For example, weather data that lacks an entity identifier and a process identifier may be automatically assigned one or more entity identifiers and/or process identifiers based on a geographic location associated with the weather data and the geographic location associated with an entity's process(es) (e.g., as indicated by an entity identifier). The data characterization module 130 may provide characterized data 118 to the data risk assessment module 140.

At step 206, the microbial risk assessment system 100 may map the characterized data to one or more risk vectors corresponding to the at least one process of the at least one entity. For each process corresponding to an entity, the risk assessment module 140 may map the microbial data 112 and/or the non-microbial data 114 to one or more risk vectors corresponding to each of the processes. Only some of the microbial data 112 and/or the non-microbial data 114 may correspond to a respective risk vector for a process, such that other data included in the microbial data 112 and/or the non-microbial data 114 may not be mapped to a respective risk vector (and may not have a resulting determined risk vector rating).

In some embodiments, the one or more risk vectors (e.g., to which the characterized data is mapped at step 206) may include at least one of: a microbial load, a rate of change of the microbial load, a microbial species, a microbial subtype, a microbial serotype, a microbial strain, comorbidities of an output corresponding to the one or more biological processes, efficacy of pathogen control programs for the one or more biological processes, environmental data for the one or more biological processes, efficiency data for the one or more biological processes, historical performance data for the one or more biological processes, economic data, management practices for the one or more biological processes, and an affiliate microbial risk rating for an affiliate entity having a business relationship with the entity. In some cases, each of the one or more risk vectors can impact a state of a biological process of the one or more biological processes.

At step 208, the microbial risk assessment system 100 may determine one or more risk vector ratings for the one or more risk vectors corresponding to the at least one process of the at least one entity based on the characterized data. Metrics, values and/or evaluations (e.g., categorical evaluations) included the characterized data 118 (e.g., the microbial data 112 and the non-microbial data 114) may be used to determine risk vector ratings for one or more respective risk vectors corresponding to a process. Techniques for determining the risk vector ratings may include those described herein. In some cases, if the microbial data 112 and/or the non-microbial data 114 includes quantitative data, the risk vector ratings may be determined based on mapping a metric included in the microbial data 112 and/or the non-microbial data 114 to one or more thresholds or ranges that are indicative of a risk vector rating or grade. As an example, for microbial data 112 corresponding to measurements of infectious disease in a livestock herd, lower levels of infectious disease may correspond to a positive (e.g., higher) risk vector rating, while higher levels of infectious disease may correspond to a negative (e.g., lower) risk vector rating. As another example, for non-microbial data corresponding to temperature for a geographic region over a period of time where products are produced, a particular range of temperatures may correspond to a positive (e.g., higher) risk vector rating, while temperatures outside the particular range may corresponding to a negative (e.g., lower) risk vector rating). In some cases, if the microbial data 112 and/or the non-microbial data 114 includes qualitative data (e.g., evaluations), the risk vector ratings or grades may be determined based on mapping an evaluation to one or more categories or partitions that are indicative of a risk vector rating or grade. As an example, for non-microbial data corresponding to an indication of a natural disaster affecting a geographic region over a period of time where crops are produced, an indication that a natural disaster did not affect a the geographic region over the period of time may correspond to a positive (e.g., higher) risk vector rating, while an indication that a natural disaster did affect a the geographic region over the period of time may corresponding to a negative (e.g., lower) risk vector rating.

In some embodiments, each of the risk vector ratings (e.g., determined at step 208) can indicate a risk of the respective risk vector to at least one of the one or more biological processes. In some cases, each of the one or more risk vector ratings can be mapped to a respective risk vector of the one or more risk vectors. In some cases, determining the one or more risk vector ratings for the one or more risk vectors can include applying a transformation (e.g., statistical analysis, normalization, weighting, etc.) to the microbial data and/or the non-microbial data to determine a first risk vector rating of the one or more risk vector ratings. In some cases, determining the one or more risk vector ratings for the one or more risk vectors can include providing the microbial data and/or the non-microbial data as an input to a model configured to determine a second risk vector rating of the one or more risk vector rating. In some cases, as described herein, the model can include a partially pooled Bayesian model. In some cases, determining the one or more risk vector ratings for the one or more risk vectors can further include determining the second risk vector rating of the one or more risk vector ratings by providing the microbial data as an input to the partially pooled Bayesian model, wherein the microbial data corresponds to and/or is compared to an expected statistical distribution. In some cases, as described herein, the model can include a categorical assessment model. In some cases, determining the one or more risk vector ratings for the one or more risk vectors can further include (ii) mapping the microbial data to one or more of a number of categorical risk tiers of the categorical assessment model, each of the plurality of categorical risk tiers corresponding to a respective weight factor and (ii) determining the second risk vector rating of the one or more risk vector ratings based on the microbial data and the weight factors. In some cases, the weight factors may be modified based on non-microbial data by applying a second weighting operation to the mapped microbial data.

In some embodiments, at least one of the microbial data and non-microbial data includes time-series data. In some cases, determining the one or more risk vector ratings for the one or more risk vectors can include generating, based on the time-series data, a time-series of risk vector ratings for the one or more risk vectors. In some cases, while not shown in FIG. 2, the method 200 may include determining a risk vector score for each of the one or more biological processes associated with the entity based on the risk vector ratings corresponding to each of the biological processes. Risk vector scores may be determined using the techniques described herein.

At step 210, the microbial risk assessment system 100 may determine a microbial risk assessment (e.g., microbial risk assessment 170) for the entity based on the one or more risk vector ratings corresponding to the at least one process. To determine the microbial risk assessment 170, the risk assessment module 140 may determine at least one risk vector score (e.g., weighted risk vector score) based on the risk vector ratings for each of the risk vectors for an entity's process. The risk vector score may be determined based on a combination of the ratings for each of the risk vectors for one of the entity's processes. In some cases, the risk assessment module 140 may determine a weighted risk vector score the ratings associated with the one or more weighted risk vectors for the particular process as described herein. As a part of the microbial risk assessment 170, the risk assessment module 140 may determine a microbial risk rating based on a combination (e.g., weighted combination) of the risk vector scores and/or the weighted risk vector scores for each of the entity's processes. If only one process corresponds to the entity, the risk assessment module 140 may determine a risk vector score or a weighted risk vector score as corresponding to the entity's microbial risk rating. A microbial risk rating may be represented by qualitative evaluations and/or a quantitative value. As an example, a microbial risk rating may be rated as any one of poor, satisfactory, good, or excellent (or any other suitable categorical expression). As another example, a microbial risk rating may be a numerical value within a standardized range of numerical values (e.g., 0-1000, 300-850, etc.).

In some embodiments, the microbial risk assessment (e.g., determined at step 210) may be indicative of a risk (e.g., financial risk, economic risk, supply chain risk, process risk, etc.) of the one or more biological processes to the entity. In some cases, determining the microbial risk assessment for the entity can further include generating, based on a time-series of risk vector ratings, a time-series of microbial risk ratings, wherein the time-series of microbial risk ratings comprises the microbial risk rating.

At step 212, the microbial risk assessment system 100 may determine an expected loss value (e.g., loss calculation estimate 180) for the entity based on the microbial risk assessment. To determine the expected loss value, the risk assessment module 100 may input the microbial risk assessment to a loss calculation model, expected loss value model, and/or any other suitable statistical model. In some cases, one or more risk vector ratings and/or risk vector scores included in the microbial risk assessment corresponding to at least one process may be provided as inputs to the model to determine the expected loss value. The expected loss value may be a numerical indicator of expected financial loss, economic loss, and/or product loss associated with at least one process corresponding to an entity.

In some embodiments, determining the expected loss value for the entity can further include providing the microbial risk rating as an input to loss calculation model configured to generate the expected loss value, where the loss calculation model includes at least one of: an unaided learning method, a Bayesian predictive function, a non-Bayesian predictive function, and an artificial intelligence technique. In some cases, a financial product can be derived from the expected loss value, wherein the financial product comprises at least one of: a risk transfer product, a credit product, a derivative product, a valuation product, and a securitization product.

In some cases, the method 200 can further include additional and/or alternative steps (not shown in FIG. 2). The method 200 can include generating a written report comprising the microbial risk assessment. The method 200 can include providing the microbial risk assessment via a user interface and/or causing sending of a message comprising the microbial risk assessment. In some cases, the method 200 can include causing sending of at least one of the microbial risk assessment or the expected loss value to an affiliate entity having a business relationship with the entity.

The microbial risk assessment method 200 may include one or more characteristics as described herein with respect to FIG. 1. For example, the microbial risk assessment and/or expected loss value for the entity (and its process) may be provided to the entity, the entity's affiliates, and/or one or more third-party entities (e.g., financial entities) for further analysis as described herein.

### Computer-Based Implementations

In some examples, some or all of the processing described above can be carried out on a personal computing device, on one or more centralized computing devices, or via cloud-based processing by one or more servers. In some examples, some types of processing occur on one device and other types of processing occur on another device. In some examples, some or all of the data described above can be stored on a personal computing device, in data storage hosted on one or more centralized computing devices, or via cloud-based storage. In some examples, some data are stored in one location and other data are stored in another location. In some examples, quantum computing can be used. In some examples, functional programming languages can be used. In some examples, electrical memory, such as flash-based memory, can be used.

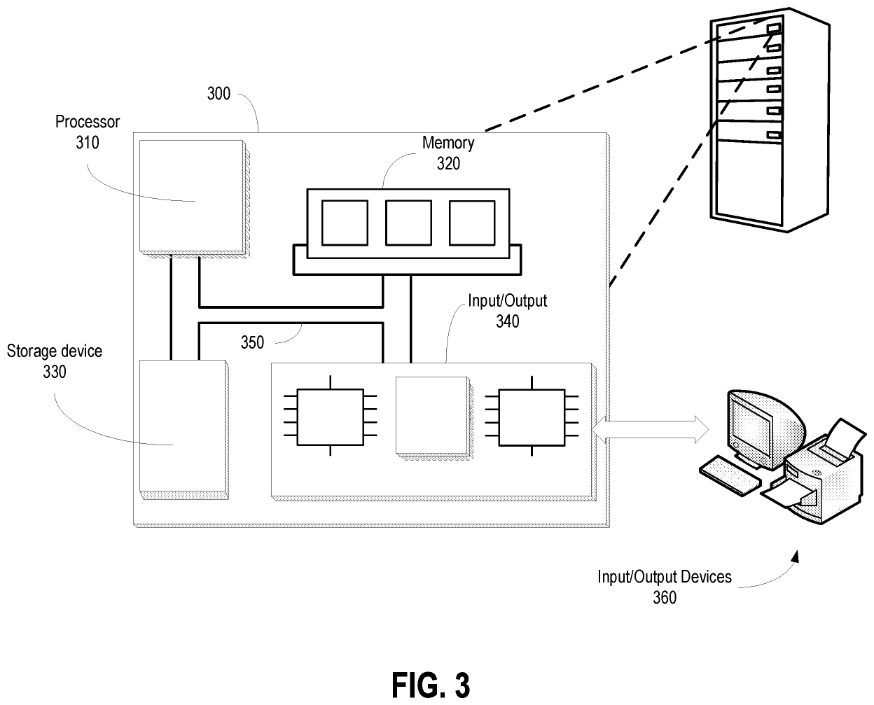

FIG. 3 is a block diagram of an example computer system 300 that may be used in implementing the technology described in this document. General-purpose computers, network appliances, mobile devices, or other electronic systems may also include at least portions of the system 300. The system 300 includes a processor 310, a memory 320, a storage device 330, and an input/output device 340. Each of the components 310, 320, 330, and 340 may be interconnected, for example, using a system bus 350. The processor 310 is capable of processing instructions for execution within the system 300. In some implementations, the processor 310 is a single-threaded processor. In some implementations, the processor 310 is a multi-threaded processor. The processor 310 is capable of processing instructions stored in the memory 320 or on the storage device 330.

The memory 320 stores information within the system 300. In some implementations, the memory 320 is a non-transitory computer-readable medium. In some implementations, the memory 320 is a volatile memory unit. In some implementations, the memory 320 is a non-volatile memory unit.

The storage device 330 is capable of providing mass storage for the system 300. In some implementations, the storage device 330 is a non-transitory computer-readable medium. In various different implementations, the storage device 330 may include, for example, a hard disk device, an optical disk device, a solid-date drive, a flash drive, or some other large capacity storage device. For example, the storage device may store long-term data (e.g., database data, file system data, etc.). The input/output device 340 provides input/output operations for the system 300. In some implementations, the input/output device 340 may include one or more of a network interface devices, e.g., an Ethernet card, a serial communication device, e.g., an RS-232 port, and/or a wireless interface device, e.g., an 802.11 card, a 3G wireless modem, or a 4G wireless modem. In some implementations, the input/output device may include driver devices configured to receive input data and send output data to other input/output devices, e.g., keyboard, printer and display devices 360. In some examples, mobile computing devices, mobile communication devices, and other devices may be used.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. The storage device 330 may be implemented in a distributed way over a network, such as a server farm or a set of widely distributed servers, or may be implemented in a single computing device.

Although an example processing system has been described in FIG. 3, embodiments of the subject matter, functional operations and processes described in this specification can be implemented in other types of digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “system” may encompass all kinds of apparatus, devices, and machines for processing data, including by way of example a programmable processor, a computer, or multiple processors or computers. A processing system may include special purpose logic circuitry, e.g., an FPGA (field programmable gate array) or an ASIC (application specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program can include, by way of example, general or special purpose microprocessors or both, or any other kind of central processing unit. Generally, a central processing unit will receive instructions and data from a read-only memory or a random access memory or both. A computer generally includes a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

### Terminology

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

What is claimed is:

1. A computer-implemented method for determining a microbial risk assessment for an entity, the method comprising:
receiving, from one or more computing systems, microbial data and non-microbial data;
identifying the microbial data and the non-microbial data as corresponding to the entity and one or more biological processes associated with the entity;
mapping the microbial data and the non-microbial data to one or more risk vectors corresponding to the one or more biological processes, wherein each of the one or more risk vectors impact a state of a biological process of the one or more biological processes;
determining, based on the microbial and non-microbial data, one or more risk vector ratings for the one or more risk vectors, each of the one or more risk vector ratings being mapped to a respective risk vector of the one or more risk vectors;
determining, based on the one or more risk vector ratings, the microbial risk assessment for the entity, wherein the microbial risk assessment comprises a microbial risk rating, wherein the microbial risk assessment is indicative of a risk of the one or more biological processes to the entity; and
determining, based on the microbial risk assessment, an expected loss value for the one or more biological processes associated with the entity.

2. The method of claim 1, wherein the microbial data comprises data collected from at least one of: direct or indirect quantification, culture, sequencing, most-probable-number, secretion assays, polymerase chain reaction (PCR), PIPER, immunoassays, whole genome sequencing, metagenomic sequencing, and next generation sequencing.

3. The method of claim 1, wherein the microbial data is sampled at a first time and a second time based on a microbial lifecycle corresponding to the one or more biological processes.

4. The method of claim 1, wherein the microbial data comprises microbial load data for Coccidia.

5. The method of claim 1, wherein the microbial data comprises microbial serotype data for Salmonella.

6. The method of claim 1, wherein the non-microbial data comprises at least one of: physical structure data for the one or more biological processes, temporal data, spatial data, business operations and financial data, imagery and remote sensing data, economic data, management practices for the one or more biological processes, sanitation practices for the one or more biological processes, pathogen control program data for the one or more biological processes, data corresponding to an output of the one or more biological processes, efficiency data for the one or more biological processes, and environmental data for the one or more biological processes.

7. The method of claim 1, wherein the one or more biological processes comprises at least one of: a food production supply chain process, a crop production process, an animal production process, a pharmaceutical production process, a chemical manufacturing process, a feed production process, and a medical device manufacturing process.

8. The method of claim 1, wherein the animal production process comprises a poultry production process.

9. The method of claim 1, wherein the one or more risk vectors comprise at least one of: a microbial load, a rate of change of the microbial load, a microbial species, a microbial subtype, a microbial serotype, a microbial strain, comorbidities of an output corresponding to the one or more biological processes, efficacy of pathogen control programs for the one or more biological processes, environmental data for the one or more biological processes, efficiency data for the one or more biological processes, historical performance data for the one or more biological processes, economic data, management practices for the one or more biological processes, and an affiliate microbial risk rating for an affiliate entity having a business relationship with the entity.

10. The method of claim 1, wherein each of the risk vector ratings indicates a risk of the respective risk vector to at least one of the one or more biological processes.

11. The method of claim 1, wherein the determining the one or more risk vector ratings for the one or more risk vectors further comprises at least one of:
(i) applying a transformation to the microbial data and/or the non-microbial data to determine a first risk vector rating of the one or more risk vector ratings; and
(ii) providing the microbial data and/or the non-microbial data as an input to a model configured to determine a second risk vector rating of the one or more risk vector rating.

12. The method of claim 11, wherein the transformation comprises a normalization technique.

13. The method of claim 11, wherein the model comprises a partially pooled Bayesian model, and wherein the method further comprises:
determining the second risk vector rating of the one or more risk vector ratings by providing the microbial data as an input to the partially pooled Bayesian model, wherein the microbial data corresponds to an expected statistical distribution.

14. The method of claim 11, wherein the model comprises a categorical assessment model, and wherein the method further comprises:
mapping the microbial data to one or more of a plurality of categorical risk tiers of the categorical assessment model, each of the plurality of categorical risk tiers corresponding to a respective weight factor; and
determining the second risk vector rating of the one or more risk vector ratings based on the microbial data and the weight factors.

15. The method of claim 14, further comprising modifying the weight factors based on non-microbial data.

16. The method of claim 1, wherein at least one of the microbial data and non-microbial data comprises time-series data, and wherein the determining the one or more risk vector ratings for the one or more risk vectors comprises:
generating, based on the time-series data, a time-series of risk vector ratings for the one or more risk vectors.

17. The method of claim 16, wherein the determining the microbial risk assessment for the entity further comprises:
generating, based on the time-series of risk vector ratings, a time-series of microbial risk ratings, wherein the time-series of microbial risk ratings comprises the microbial risk rating.

18. The method of claim 1, wherein the determining the expected loss value for the entity further comprises:
providing the microbial risk rating as an input to loss calculation model configured to generate the expected loss value, where the loss calculation model comprises at least one of: an unaided learning method, a Bayesian predictive function, a non-Bayesian predictive function, and an artificial intelligence technique.

19. The method of claim 1, wherein a financial product is derived from the expected loss value, wherein the financial product comprises at least one of: a risk transfer product, a credit product, a derivative product, a valuation product, and a securitization product.

20. The method of claim 1, further comprising:
generating a written report comprising the microbial risk assessment.

21. The method of claim 1, further comprising at least one of:
(i) providing the microbial risk assessment via a user interface; and
(ii) causing sending of a message comprising the microbial risk assessment.

22. The method of claim 1, further comprising:
causing sending of at least one of the microbial risk assessment or the expected loss value to an affiliate entity having a business relationship with the entity.

23. A system for determining a microbial risk assessment for an entity, the system comprising:
one or more computing devices programmed to perform operations comprising:
receiving and identifying microbial data and non-microbial data as corresponding to the entity and one or more biological processes associated with the entity;
mapping the microbial data and the non-microbial data to one or more risk vectors corresponding to the one or more biological processes, wherein each of the one or more risk vectors impact a state of a biological process of the one or more biological processes;
determining, based on the microbial and non-microbial data, one or more risk vector ratings for the one or more risk vectors, each of the one or more risk vector ratings being mapped to a respective risk vector of the one or more risk vectors;
determining, based on the one or more risk vector ratings, the microbial risk assessment for the entity, wherein the microbial risk assessment comprises a microbial risk rating determined based on the one or more risk vector ratings, wherein the microbial risk assessment is indicative of a risk of the one or more biological processes to the entity; and
determining, based on the microbial risk assessment, an expected loss value for the one or more biological processes associated with the entity.
